(19) United States
(12) Patent Application Publication
(10) Pub. No.: US 2016/0051542 A1
(43) Pub. Date: Feb. 25, 2016

(54) METHODS OF ADMINISTRATION OF SINGLE DOSES OF VANOXERINE TO TERMINATE ACUTE EPISODES OF CARDIAC ARRHYTHMIA

(71) Applicant: CHANRX CORPORATION, Cleveland, OH (US)

(72) Inventor: Arthur M. Brown, Brecksville, OH (US)

(21) Appl. No.: 14/781,298

(22) PCT Filed: Apr. 25, 2014
(86) PCT No.: PCT/US2014/035574

§ 371 Date: Sep. 29, 2015

Related U.S. Application Data
(60) Provisional application No. 61/915,792, filed on Dec. 13, 2013.
(60) Provisional application No. 61/905,054, filed on Nov. 15, 2013.
(60) Provisional application No. 61/816,214, filed on Apr. 26, 2013.

(51) Int. Cl. A61K 31/495

(52) U.S. Cl. CPC A61K 31/495

(57) ABSTRACT

Compositions and methods of treating patients suffering from symptoms of recent onset atrial fibrillation or atrial flutter comprising administration of a single dose of about 200 to about 400 mg of vanoxerine to return said patient to normal sinus rhythm in less than about 24 hours.

### FIELD OF THE INVENTION

Presently disclosed embodiments are related to compositions comprising vanoxerine and methods of treatment comprising administration of vanoxerine to a mammal for terminating acute episodes of cardiac arrhythmia. Presently disclosed embodiments particularly relate to methods for dosing and treatment methodologies for administration of vanoxerine in the case of terminating episodes of cardiac arrhythmia in a single dose.

### BACKGROUND

Vanoxerine (1-[2-[bis(4-fluorophenyl)methoxy]ethyl]-4-(3-phenylpropyl)piperazine), its manufacture and/or certain pharmaceutical uses thereof are described in U.S. Pat. No. 4,202,896, U.S. Pat. No. 4,476,129, U.S. Pat. No. 4,874,765, U.S. Pat. No. 6,743,797 and U.S. Pat. No. 7,700,600, as well as European Patent EP 243,903 and PCT International Application WO 91/01732, each of which is incorporated herein by reference in its entirety.

Vanoxerine has been used for treating cocaine addiction, acute effects of cocaine, and cocaine cravings in mammals, as well as dopamine agonists for the treatment of Parkinsonism, acromegaly, hyperprolactinemia and diseases arising from a hypofunction of the dopaminergic system. (See U.S. Pat. No. 4,202,896 and WO 91/01732). Vanoxerine has also been used for treating and preventing cardiac arrhythmia in mammals. (See U.S. Pat. No. 6,743,797 and U.S. Pat. No. 7,700,600).

It is desirable to optimize compositions comprising vanoxerine for treatment of cardiac arrhythmia and methods of treatment using vanoxerine in single doses to arrest cardiac arrhythmia in particular atrial fibrillation and atrial flutter in a patient.

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. In the US alone, AF currently afflicts more than 2.3 million people. By 2050, it is expected that there will be more than 12 million individuals afflicted with AF. While AF is rarely a lethal arrhythmia, it is responsible for substantial morbidity and can lead to complications such as the development of congestive heart failure or thromboembolism. Currently available Class I and Class III anti-arrhythmic drugs reduce the rate of re-occurrence of AF, but are of limited use because of a variety of potentially adverse effects, including ventricular proarrhythmia. Because current therapy is inadequate and fraught with side effects, there is a clear need to develop new therapeutic approaches.

Current first line pharmacological therapy options for AF include drugs for rate control. Despite results from several studies suggesting that rate control is equivalent to rhythm control, many clinicians believe that patients are likely to have better functional status when in sinus rhythm. Further, being in AF may introduce long-term mortality risk, where achievement of rhythm control may improve mortality.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, these agents have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Prior studies have been performed using single dose administration of flecainide or propafenone (Class I drugs) in terminating atrial fibrillation. Particular studies investigated the ability to provide patients with a known dose of one of the two drugs so as to self-medicate should cardiac arrhythmia occur. P. Alboni, et al., “Outpatient Treatment of Recent-Onset Atrial Fibrillation with the ‘Pill-in-the-Pocket’ Approach,” NEJM 351; 23 (2004); L. Zhou, et al., “‘A Pill in the Pocket’ Approach for Recent Onset Atrial Fibrillation in a Selected Patient Group,” Proceedings of UCLA Healthcare 15 (2011). However, the use of flecainide and propafenone has been criticized as including candidates having structural heart disease and thus providing patients likely to have risk factors for stroke who should have received antithrombotic therapy, instead of the flecainide or propafenone. NEJM 352:11 (Letters to the Editor) (Mar. 17, 2005). Similarly, the use of warfarin concomitantly with propafenone was criticized.

Anti-arrhythmic agents of Class III are drugs that cause a selective prolongation of the action potential duration (APD) without a significant depression of the maximum upstroke velocity (Vₘₐₓ). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A Third Class of Anti-Arrhythmic Action: Effects on Atrial and Ventricular Intracellular Potentials and other Pharmacological Actions on Cardiac Muscle of MJ 1999 and AH 3747,” (Br. J. Pharmacol 39:675-689 (1970), and Singh B. N., Vaughan Williams E. M., “The Effect of Amiodarone, a New Anti-Anginal Drug, on Cardiac Muscle,” Br. J. Pharmacol 39:657-667 (1970)), but these are not selective Class III agents. Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey,” J. Am. Coll. Cardiol. 20:1063-1065 (1992)). Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na+ or Ca²+ currents; hereinafter I_{Na} and I_{Ca}, respectively) or by reducing outward repolarizing potassium K+ currents. The delayed rectifier (I_{K}) K+ current is the main outward current involved in the overall repolarization process during the action potential plateau, whereas the transient outward (Iₜₒ) and inward rectifier (I_{KI}) K+ currents are responsible for the rapid initial and terminal phases of repolarization, respectively.

Cellular electrophysiologic studies have demonstrated that I_{K} consists of two pharmacologically and kinetically distinct K+ current subtypes, I_{Kr} (rapidly activating and deactivating) and I_{Ks} (slowly activating and deactivating). (Sanguinetti and Jurkiewicz, “Two Components of Cardiac Delayed Rectifier K+ Current. Differential Sensitivity to Block by Class III Anti-Arrhythmic Agents,” J Gen Physiol 96:195-215 (1990)). I_{Kr} is also the product of the human ether-a-go-go gene (hERG). Expression of hERG cDNA in cell lines leads to production of the hERG current which is almost identical to I_{Kr} (Curran et al., “A Molecular Basis for Cardiac Arrhythmia: hERG Mutations Cause Long QT Syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2, 4′-piperidin]-6yl], (+)−, monochloride (MK-499) predominantly, if not exclusively, block I_{Kr}. Although amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, as a nonspecific adrenergic blocker, acts as an inhibitor of the enzyme phospholipase, and causes pulmonary fibrosis (Nademanee, K., “The Amiodarone Odessey.” J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness, by prolonging APD, prevents and/or terminates reentrant arrhythmias. Most selective Class III antiarrhythmic agents currently in development, such as d-sotalol and dofetilide predominantly, if not exclusively, block I_{Kr}, the rapidly activating component of I_{K} found both in atria and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardiographic QT interval, hence termed “acquired long QT syndrome,” has been observed when these compounds are utilized (Roden, D. M., “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S17-S22). The pro-arrhythmic tendency led to suspension of the SWORD trial when d-sotalol had a higher mortality than placebo controls.

The slowly activating component of the delayed rectifier (I_{Ks}) potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supra-ventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced Effectiveness and Dangers of Reverse use Dependence,” Circulation, 81:686-690 (1990); Sadanaga et al., “Clinical Evaluation of the Use-Dependent QRS Prolongation and the Reverse Use-Dependent QT Prolongation of Class III Anti-Arrhythmic Agents and Their Value in Predicting Efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-Dependent Prolongation of Cardiac Action Potentials by a Methanesulfonanilide Class III Anti-Arrhythmic Agent: Specific Block of Rapidly Activating Delayed Rectifier K+current by Dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

Vanoxerine has been indicated for treatment of cardiac arrhythmias. Indeed, certain studies have looked at the safety profile of vanoxerine and stated that no side-effects should be expected with a daily repetitive dose of 50 mg of vanoxerine. (U. Sogaard, et. al., “A Tolerance Study of Single and Multiple Dosing of the Selective Dopamine Uptake Inhibitor GBR 12909 in Healthy Subjects,” International Clinical Psychopharmacology, 5:237-251 (1990)). However, Sogaard, et. al. also found that upon administration of higher doses of vanoxerine, some effects were seen with regard to concentration difficulties, increase systolic blood pressure, asthenia, and a feeling of drug influence, among other effects. Sogaard, et. al. also recognized that there were unexpected fluctuations in serum concentrations with regard to these healthy patients. While they did not determine the reasoning, control of such fluctuations may be important to treatment of patients.

Further studies have looked at the ability of food to lower the first-pass metabolism of lipophilic basic drugs, such as vanoxerine. (S. H. Ingwersen, et. al., “Food Intake Increases the Relative Oral Bioavailability of Vanoxerine,” Br. J. Clin. Pharmac; 35:308-130 (1993)). However, no methods have been utilized or identified for treatment of cardiac arrhythmias in conjunction with the modulating effects of food intake.

Therefore, it is necessary to develop compositions comprising vanoxerine and methods of administration of the same for safe and fast termination of atrial fibrillation (AF) and atrial flutter (AFL), including patients suffering from recent onset of AF or AFL.

### SUMMARY

Embodiments of the present disclosure relate to methods for treating a mammal with recent onset symptomatic AF or AFL comprising: administering a composition comprising vanoxerine to a mammal to restore normal sinus rhythm in less than about 8 hours.

A further embodiment of the present disclosure relates to a method for restoring a mammal, with recent onset symptomatic AF or AFL, to normal sinus rhythm in less than about 8 hours comprising: administering a composition comprising 200 to 400 mg of vanoxerine and a pharmaceutical carrier to said mammal.

A further embodiment of the present disclosure relates to a method for restoring a mammal, having symptomatic AF or AFL for less than about 24 hours, to normal sinus rhythm in less than about 24 hours comprising: administering a composition comprising vanoxerine and a pharmaceutical carrier to said mammal.

A further embodiment of the present disclosure relates to a method for restoring a mammal, having symptomatic AF or AFL for less than about 72 hours, to normal sinus rhythm in less than about 24 hours comprising: administering a composition comprising 200 to 400 mg of vanoxerine and a pharmaceutical carrier to said mammal.

A method of treating a patient having recent onset of AF or AFL comprising administration of a single dose of a pharmaceutical composition comprising about 200 to about 400 mg of vanoxerine, and wherein said patient is converted to normal sinus rhythm at a rate at least 33% greater than the rate of conversion as compared to placebo.

A method for restoring normal sinus rhythm to a patient suffering from recent onset symptomatic atrial fibrillation or atrial flutter in less than about 24 hours by administering to said patient at least 200 mg of vanoxerine.

A method of treating a patient having recent onset of AF or AFL comprising administration of a single dose of a pharmaceutical composition comprising about 200 to about 400 mg of vanoxerine, and wherein said patient is converted to normal sinus rhythm at a rate of at least 50% better than conversion as compared to placebo at a time period of 0-4 hours.

A method of treating a patient suffering from symptoms of atrial fibrillation or atrial flutter for less than about 72 hours comprising administration of about 200 to about 400 mg of vanoxerine.

A method for terminating atrial flutter or atrial fibrillation comprising: administering a first dose of at least 200 mg of vanoxerine to a patient to terminate said atrial flutter or atrial fibrillation in less than 24 hours; administering a subsequent doses of an effective amount of vanoxerine to achieve steady-state status of vanoxerine in the patient; and administering of an effective amount of vanoxerine to maintain a steady-state status of vanoxerine in the patient.

### BRIEF DESCRIPTION OF THE FIGURES

FIG. 1 depicts a chart showing the percent conversion to normal sinus rhythm over time.

### DETAILED DESCRIPTION OF PREFERRED EMBODIMENTS

All references cited herein are hereby incorporated by reference in their entirety.

As used herein, the term “about” is intended to encompass a range of values±10% of the specified value(s). For example, the phrase “about 20” is intended to encompass±10% of 20, i.e. from 18 to 22, inclusive.

As used herein, the term “vanoxerine” refers to vanoxerine and pharmaceutically acceptable salts thereof.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, the term “subject” refers to a warm blooded animal such as a mammal, preferably a human or a human child, which is afflicted with, or has the potential to be afflicted with one or more diseases and conditions described herein.

As used herein, “therapeutically effective amount” refers to an amount which is effective in reducing, eliminating, treating, preventing or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either vanoxerine or a pharmaceutically acceptable composition comprising vanoxerine.

As used herein, “CYP3A4” means the cytochrome P450 3A4 protein, which is a monooxygenase that is known for its involvement in drug metabolism.

As used herein, “administering” or “administer” refers to the actions of a medical professional or caregiver, or alternatively self-administration by the patient.

As used herein, “recent onset” means between 3 hours and 7 days.

The term “steady state” means wherein the overall intake of a drug is fairly in dynamic equilibrium with its elimination.

As used herein, a “pre-determined” plasma level or other physiological tissue or fluid and refers to a concentration of vanoxerine at a given time point. Typically, a pre-determined level will be compared to a measured level, and the time point for the measured level will be the same as the time point for the pre-determined level. In considering a pre-determined level with regard to steady state concentrations, or those taken over a period of hours, the pre-determined level is referring to the mean concentration taken from the area under the curve (AUC), as the drug increases and decreases in concentration in the body with regard to the addition of a drug pursuant to intake and the elimination of the drug via bodily mechanisms.

Cardiac arrhythmias include atrial, junctional, and ventricular arrhythmias, heart blocks, sudden arrhythmic death syndrome, and include bradycardias, tachycardias, re-entrant, and fibrillations. These conditions, including the following specific conditions: atrial flutter, atrial fibrillation, multifocal atrial tachycardia, premature atrial contractions, wandering atrial pacemaker, supraventricular tachycardia, AV nodal reentrant tachycardia, junctional rhythm, junctional tachycardia, premature junctional contraction, premature ventricular contractions, ventricular bigeminy, accelerated idioventricular rhythm, monomorphic ventricular tachycardia, polymorphic ventricular tachycardria, and ventricular fibrillation, and combinations thereof are all capable of severe morbidity and death if left untreated. Methods and compositions described herein are suitable for the treatment of these and other cardiac arrhythmias.

Interestingly, studies have identified that human subjects have significant variability with regard to the metabolism of vanoxerine. Vanoxerine is susceptible to metabolism by CYP3A4 among other known P450 cytochromes. Accordingly, the bioavailability of a given dose of vanoxerine is impacted by certain P450 cytochromes. In particular, studies have identified that human subjects have variability with regard to metabolism which is predicted to be based on CYP3A4 and other P450 cytochromes. Typically, patients fall within one of two groups, a fast metabolism or a slow metabolism, such that the patients can be grouped with other patients and will have similar metabolic profiles for a given dose of vanoxerine. Patients in the fast metabolism group respond differently to vanoxerine than patients in the slow metabolism group with regard to Cₘₐₓ, tₘₐₓ, and AUC plasma concentrations as well as the half-life. Accordingly, it is possible to define whether a given patient is a fast or a slow metabolizer and predict their pharmacokinetic response to vanoxerine. Accordingly, determination of the patient's status within the fast or slow metabolic group can be utilized for improving efficacy and treatment of a patient.

Additionally, patients fall within a gradient within the slow and fast metabolism groups. Accordingly, there exists, even within the groupings, a continuum that provides that some people are faster or slower metabolizers even within the groups. Additional factors also play into the variability with regard to patient populations. Accordingly, when providing efficacious treatment for termination of cardiac arrhythmias, in some embodiments, it is important to determine or recognize where the patient falls within the spectrum of vanoxerine bioavailability, and provide a dose of vanoxerine that will be efficacious for that patient while also maximizing the safety profile of the drug.

Vanoxerine also has a moderately low oral bioavailability as a result of incomplete absorption and substantial first pass metabolism, from CYP3A4 and other p450 inhibitors. Vanoxerine is primarily eliminated from the body in urine, bile, and feces. Indeed, a substantial amount of the drug is expelled unabsorbed into the feces. Additionally, pharmacokinetic parameters from tests in dogs suggest that there is a slow Tₘₐₓ of about 3 hours, low systemic bioavailability (23%) and slow elimination from the plasma (T_{1/2} of 22 hours). However, the long half-life of the drug may actually be utilized to minimize the continuous or regular dosing of the drug.

Further studies have questioned whether sustained, and/or chronic use of vanoxerine is suitable for mammalian patients. Preliminary studies have suggested that daily use of a drug over 7, 10, and 14 days may lead to increased heart rate and systolic blood pressure when taking concentrations of 75, 100, 125, and 150 mg of vanoxerine a day. However, control and prevention of events of cardiac arrhythmia are important to these patients to prevent future re-occurrences and the deleterious effects and morbidity.

Indeed, control and prevention of events of cardiac arrhythmia are important to these patients to prevent future re-occurrences and the deleterious effects and morbidity. One issue is that cardiac arrhythmia is a progressive disease and patients who suffer from a first cardiac arrhythmia are pre-disposed to suffering from additional episodes of cardiac arrhythmia. Any cardiac arrhythmia involves risk with regard to mortality and morbidity, and so terminating the cardiac arrhythmia in a timely and safe manner is a critical need for these patients. Therefore, preventing further arrhythmic events is paramount for limiting this risk.

Therefore, upon an occurrence of cardiac arrhythmia, patients often visit an emergency room or other medical provider for administration of certain drugs that treat the cardiac arrhythmia, or other treatments, including ablation. However, it is not always feasible to quickly reach a doctor for fast, safe, and effective treatment of cardiac arrhythmia. Furthermore, in view of the dangers of some concurrent medications with other drugs for treatment of cardiac arrhythmias, it is advantageous to provide patients who have previously suffered from a cardiac arrhythmia, and have successfully treated that cardiac arrhythmia with a single-dose of vanoxerine, whether in a hospital, emergency room, a rescue vehicle, or as provided for self-administration for returning the patient to normal sinus rhythm.

Additional concerns for patients who have suffered from cardiac arrhythmia are compounding heart disease, as well as angina pectoris as well as other heart pain, chest pain, and other complications. Typically, concomitant use of an atrial fibrillation drug with a number of other drugs is contraindicated because of any number of interactions between the two drugs. However, certain drugs may establish a beneficial co-administration with vanoxerine wherein the concomitant administration of vanoxerine and at least one additional drug for treatment of cardiac arrhythmia allows for maintenance of steady state status of vanoxerine while providing for more frequent administration of said at least one additional drug. The combination allows for regular administration of vanoxerine to maintain normal sinus rhythm, but without the need for daily maintenance therapy, while providing for a dose of a second drug to be taken more frequently than the vanoxerine, to aiding the maintenance of normal sinus rhythm, and preventing further episodes of cardiac arrhythmia.

Accordingly, it is advantageous to provide a composition comprising vanoxerine in an amount sufficient to restore normal sinus rhythm within 24 hours of administration of the composition, and preferably within 12 or 8 hours of administration. Accordingly, the patient can quickly return to normal sinus rhythm without the need for ablation of other invasive techniques or by simply waiting to see if the AF or AFL will subside on its own over time.

It is further advantageous to provide a method for administration wherein a patient suffering from recent onset of atrial fibrillation or atrial flutter (less than an hour to about 7 days), is administered a single dose of vanoxerine of between 200 to 400 mg to return the patient to normal sinus rhythm within 24 hours.

In some embodiments, a method for administration of vanoxerine to a patient suffering from onset of atrial fibrillation or atrial flutter from between about 3 and about 24 hours, is administered a single dose of vanoxerine of between 200 to 400 mg to return about 60% of patients to normal sinus rhythm in under 8 hours and about 80% of patients to normal sinus rhythm in about 24 hours. In preferred embodiments, about 90% of patients convert to normal sinus rhythm in less than about 24 hours.

In further embodiments, the method comprises administration of a single dose of vanoxerine to a patient suffering from atrial fibrillation or atrial flutter for less than about 24 hours, wherein said single dose of vanoxerine returns said patient to normal sinus rhythm in less than about 24 hours.

Other embodiments comprise patients having onset of AF or AFL in less than about 8 hours, 12 hours, 24 hours, 36 hours, 48 hours, 72 hours, and 96 hours wherein a single dose of vanoxerine is administered and is effective in returning said patient to normal sinus rhythm in less than about 24 hours. Further embodiments convert patients to normal sinus rhythm in less than about 4 hours, or less than about 8 hours from a single dose of vanoxerine given to said patient.

It is further conceived that because of the long half-life of vanoxerine, it may be advantageous to administer an initial dose of vanoxerine to restore normal sinus rhythm in less than 24 hours followed by administration of vanoxerine to create steady state in the patient, and a maintenance phase comprising a reduced dosing schedule, such as every 24, 48, 72, 96 hours, or more, based on the extended half-life of the vanoxerine, so as to maintain steady state. By maintaining the concentration in the patient at an efficacious level, the patient is less likely to recess back into arrhythmia. Accordingly, the method supports the swift return to normal sinus rhythm and includes a dosing regimen that supports prevention or re-occurrence of arrhythmia.

Accordingly, a method comprises administration of a single dose to a patient having onset of AF or AFL in less than about 24 hours comprising administration of a single dose of a pharmaceutical composition comprising about 200 to about 400 mg of vanoxerine, and wherein said patient is converted to normal sinus rhythm in less than about 24 hours followed by administration of an effective amount of vanoxerine to induce steady state and followed by a maintenance phase to maintain said steady state.

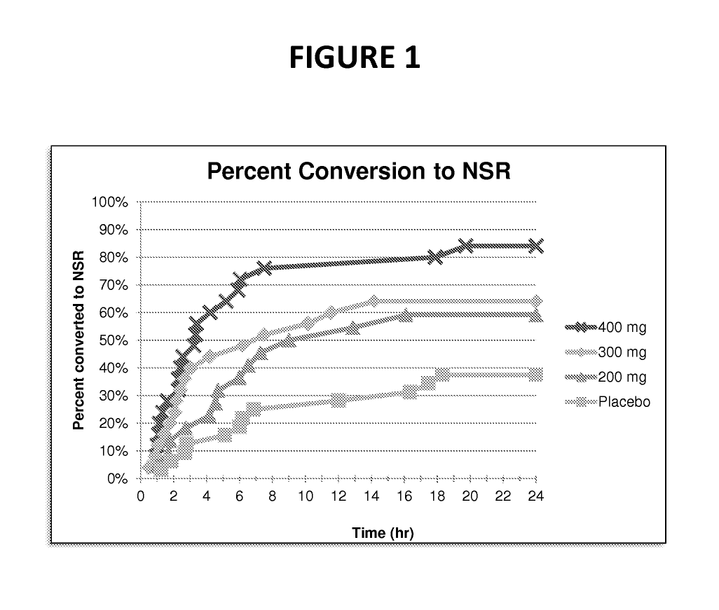

In view of FIG. 1, and as shown in Tables 1, 2, and 3, after administration of a placebo, the rate of conversion is approximately linear after about 4-6 hours, with only about an additional 10% of patients returning to normal sinus rhythm over the period of 6 hours to 24 hours, as shown in FIG. 1. Wherein at a time of about 4 hours, conversion from placebo is 13%, at 6 hours at about 20%, at 8 hours at about 23%, at 12 hours, about 30%, at 16 hours at about 33%, and at 24 hours at about 38%.

However, as further depicted in FIG. 1, non-placebo patients, have a much faster conversion to normal sinus rhythm in the first 0-8 hours, before similarly tapering off to a slightly linear increase in conversion from a time of about 8 hours to about 24 hours. However, doses of 300 and 400 mg have a rate of return to normal sinus rhythm in at 4 hours of 40% and 52% respectively, whereas placebo is a rate of conversion at 13%. Even doses of 200 mg show substantial improvement over placebo at 4 hours at 18%, and continues to 45% conversion at a time of about 8 hours, as compared to conversion of 23% at 8 hours for placebo, a nearly two fold increase even at the 200 mg dose.

Accordingly, a method comprises administration of a single dose to a patient having recent onset of AF or AFL comprising administration of a single dose of a pharmaceutical composition comprising about 200 to about 400 mg of vanoxerine, and wherein said patient is converted to normal sinus rhythm at a rate at least 33% greater than that of conversion as compared to placebo at the same time. In other embodiments, the rate of conversion is at least 50%, 66%, 75%, 100%, 150%, and 200% greater than the rate of conversion as compared to placebo.

In some embodiments, a dosage of 1 mg to 1000 mg per unit dose is appropriate. Other embodiments may utilize a dosage of about 50 mg to 800 mg, or about 25 to 100 mg, or about 100 mg to about 600 mg, or about 200 to about 400 mg. Preferred embodiments include administration of vanoxerine in about 200, 300, or 400 mg for the initial dose to return said patient to normal sinus rhythm. Subsequent 25, 50, 75, 100, 125, 150, 200, 300, and 400 mg doses for daily dosing or a loading period and for maintenance amounts for treatment of chronic cardiac arrhythmia are suitable in further embodiments.

In treating a patient experiencing recent onset of AF or AFL, target plasma level concentrations, taken at a time point of 1 hour post administration are about 5 to about 1000 ng/ml. In alternative embodiments, physiological concentrations, as measured in the plasma at a time of 1 hour post administration are about 20 to about 400 ng/ml, or about 20 to about 200 ng/ml, or about 25 to about 150 ng/ml or about 40 to about 125 ng/ml, or about 60 to about 100 ng/ml. In some cases it may be necessary to test plasma levels to confirm an efficacious concentration is met to return the patient to normal sinus rhythm. If efficacious concentrations are not met, a further dose may be administered to achieve an efficacious level in the body to reach normal sinus rhythm.

In measuring plasma levels for confirmation of half-life and/or steady state plasma levels, it may be necessary to take additional measurements at further time points, such as 2, 4, 6, 8, 12, 24, 36, 48, 72, hours, and other times as appropriate. In some cases, it may be advantageous to test plasma levels every 24, 48, 72, or 96 hours, or to test plasma levels prior to or subsequent to a further administration of vanoxerine.

Accordingly, in some embodiments, it is advantageous to provide a first initial dose of vanoxerine to treat AF/AFL comprising administration of about 200 to about 400 mg to restore normal sinus rhythm in at least about 24 hours. Upon occurrence of normal sinus rhythm, a further loading phase of vanoxerine, wherein a patient is given one or more doses of vanoxerine for about 3 to about 14 days to reach a steady state (as measured in plasma or some other bodily fluid) concentrations of vanoxerine for restoration or maintenance of normal sinus rhythm in a mammal, and finally comprising administration of vanoxerine to maintain said steady state plasma level in said patient to prevent re-occurrence of AF/AFL, in a maintenance phase. Suitable maintenance phases include regular dosing schedules of an effective amount of vanoxerine to maintain the steady-state plasma level in the patient. In particular embodiments, the steady state levels are a mean plasma concentration of about 1 to about 200 ng/ml, about 5 to about 200 ng/ml, about 10 to about 200 ng/ml, about 20 to about 150 ng/ml, about 25 to about 125 ng/ml.

In some embodiments, maintenance of a predetermined plasma level is achieved through dosing where the vanoxerine drug is administered once a day, once every other day, once every third day, once every 4^{th}, 5^{th}, 6^{th}, and 7^{th} days, wherein an additional drug is administered between vanoxerine administrations. Vanoxerine has a relatively long plasma half-life of about 22 hours, and further tests suggest that repetitive dosing in dogs provides a half-life that is considerably longer at about 66 hours. Furthermore, steady state plasma levels are achieved within 3 days of oral dosing in some studies and up to 14 days in other studies. In human studies, the typical time is between about 3-11 days for reaching steady state status. Indeed, tests on recovery of administration of radioactivity labeled vanoxerine in rats was incomplete. This, coupled with the observed biliary excretion, suggests enterohepatic circulation may be occurring. This provides for an opportunity to achieve steady state plasma levels for restoration or maintenance of normal sinus rhythm in mammals.

Therefore, it may be advantageous to further utilize a method of loading vanoxerine to achieve and maintain a steady state in connection with an additional pharmaceutical composition, wherein the vanoxerine is first administered to a mammal to reach a predetermined plasma level, upon reaching such plasma level at a pre-determined point subsequent to the vanoxerine administration, vanoxerine can then be administered to maintain said pre-determined plasma level about every 66 hours, or at a rate determined in the individual patient, because of the long half-life, which is further increased by steady state.

In other embodiments, it is advantageous to provide for a certain dose, or a maximum dose at a given time point after administration of the vanoxerine to safely and effectively treat the cardiac arrhythmia. Accordingly, modification of Cₘₐₓ and tₘₐₓ is appropriate to maintain consistent Cₘₐₓ plasma level concentrations for a particular patient. Cₘₐₓ concentration is about 5 to about 1000 ng/ml. In alternative embodiments, plasma level concentrations at 1 hour post administration are about 10 to about 400 ng/ml, or about 20 to about 200 ng/ml, or about 20 to about 150 ng/ml, or about 25 to about 125 ng/ml or about 40 to about 100 ng/ml, and about 60 to about 100 ng/ml. Conversely tₘₐₓ is appropriately reached at about 1 hour post administration. In other embodiments, tₘₐₓ is appropriately reached at about 30 minutes, or about 90 minutes, or about 120 minutes, or about 240 minutes post administration. These maximum values vary widely by patient and modification of the dose, of the dosing schedule, of diet, and of other concomitant medications may be utilized to reach a predetermined therapeutic level.

In other embodiments and methods of administration, an initial dose, a loading phase, and a maintenance phase may all be administered via different mechanisms. For example, a patient may be administered an initial dose in IV or as a parenteral bolus injection. The loading phase may be via an infusion device, either implanted or carried with the patient, and the maintenance phase may be with an oral formulation. The particular mode of administration, accordingly, may be altered in one or more of the phases as is appropriate for the particular patient and treatment scenario.

Suitable methods for treatment of cardiac arrhythmias include various dosing schedules which may be administered by any technique capable of introducing a pharmaceutically active agent to the desired site of action, including, but not limited to, buccal, sublingual, nasal, oral, topical, rectal and parenteral administration. Dosing may include single daily doses, multiple daily doses, single bolus doses, slow infusion injectables lasting more than one day, extended release doses, IV or continuous dosing through implants or controlled release mechanisms, and combinations thereof. These dosing regimens in accordance with the method allow for the administration of the vanoxerine in an appropriate amount to provide an efficacious level of the compound in the blood stream or in other target tissues. Delivery of the compound may also be through the use of controlled release formulations in subcutaneous implants or transdermal patches.

For oral administration, a suitable composition containing vanoxerine may be prepared in the form of tablets, dragees, capsules, syrups, and aqueous or oil suspensions. The inert ingredients used in the preparation of these compositions are known in the art. For example, tablets may be prepared by mixing the active compound with an inert diluent, such as lactose or calcium phosphate, in the presence of a disintegrating agent, such as potato starch or microcrystalline cellulose, and a lubricating agent, such as magnesium stearate or talc, and then tableting the mixture by known methods.

Tablets may also be formulated in a manner known in the art so as to give a sustained release of vanoxerine. Such tablets may, if desired, be provided with enteric coatings by known method, for example by the use of cellulose acetate phthalate. Suitable binding or granulating agents are e.g. gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing vanoxerine and at least one diluent, and optionally a part of the disintegrating agent, is granulated together with an aqueous, ethanolic or aqueous-ethanolic solution of the binding agents in an appropriate equipment, then the granulate is dried. Thereafter, other preservative, surface acting, dispersing, disintegrating, gliding and anti-adhesive additives can be mixed to the dried granulate and the mixture can be compressed to tablets or capsules.

Tablets may also be prepared by the direct compression of the mixture containing the active ingredient together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, vanoxerine and the desired additives may be filled into a capsule, such as a hard or soft gelatin capsule. The contents of a capsule and/or caplet may also be formulated using known methods to give sustained release of the active compound.

Liquid oral dosage forms of vanoxerine may be an elixir, suspension and/or syrup, where the compound is mixed with a non-toxic suspending agent. Liquid oral dosage forms may also comprise one or more sweetening agent, flavoring agent, preservative and/or mixture thereof.

For rectal administration, a suitable composition containing vanoxerine may be prepared in the form of a suppository. In addition to the active ingredient, the suppository may contain a suppository mass commonly used in pharmaceutical practice, such as Theobroma oil, glycerinated gelatin or a high molecular weight polyethylene glycol.

For parenteral administration, a suitable composition of vanoxerine may be prepared in the form of an injectable solution or suspension. For the preparation of injectable solutions or suspensions, the active ingredient can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspensions may be prepared from sterile powders or granules having one or more carriers or diluents mentioned for use in the formulations for oral administration. Parenteral administration may be through intravenous, intradermal, intramuscular or subcutaneous injections.

### EXAMPLES

The materials, methods, and examples presented herein are intended to be illustrative, and not to be construed as limiting the scope or content of the invention. Unless otherwise defined, all technical and scientific terms are intended to have their art-recognized meanings.

### Example 1

3 different cohorts, each including 35 subjects were enrolled in a study with 25 taking vanoxerine and 10 receiving placebo. Cohort 1 included 200 mg vanoxerine, Cohort 2 include 200 or 300 mg of vanoxerine, and Cohort 3 included 200, 300, or 400 mg vanoxerine. The vanoxerine or identical appearing placebo was randomly assigned and administered in a double-blinded fashion.

Inclusion criteria included: male or female over the age of age, symptomatic AF/AFL for more than 3 hours and less than 7 days, as dated by symptoms, and adherence to local clinical standards or the ACC/ACA/ESC practice guidelines for AF/AFL regarding thromboembolic event prevention and treatment

Exclusion Criteria Included:
(a) Systolic blood pressure <100 mmHG, HR <50 bpm
(b) Average QTcF >440 ms, WRS interval >140 ms
(c) Paced atrial or ventricular rhythm
(d) Serum potassium <3.5 meq/L
(e) History of receiving another Class 1 or Class III antiarrhythmic drug within 3 days of randomization, amiodarone (oral or IV) within 3 months
(f) Acute coronary syndrome within 30 days prior to randomization
(g) Aortic stenosis with AVA ≦1.0 cm²
(h) Mitral stenosis with MVA of <1.5 cm²
(i) Acute pulmonary edema/embolism
(j) Stroke within 30 days or TIA within 48 hours
(k) Untreated hyperthyroidism
(l) Acute pericarditis
(m) Postoperative AF/AFL within 7 days
(n) History of failed Direct current cardioversion
(o) History of polymorphic ventricular tachycardia (e.g. Torsade de Pointes)
(p) History or family history of long QT syndrome
(q) History of ventricular tachycardia requiring drug or device therapy
(r) History of NYHA Heart Failure Class III or IV or recent (within 1 month) onset of heart failure not related to rapid ventricular response AF
(s) Ejection fraction of 35% or less
(t) History of prior ablation therapy for cardiac arrhythmias

Statistical Data:

4-Hour Efficacy Endpoints:
(a) the proportion of subjects who convert to sinus rhythm for at least 1 minute through 4 hours after start of study drug.
(b) the proportion of subjects in sinus rhythm at 4 hours after start of study drug
(c) time to restoration of sinus rhythm within 4 hours

24-Hour Efficacy Endpoints
(a) the proportion of subjects who convert to sinus rhythm for at least 1 minute through 24 hours after start of study drug.
(b) the proportion of subjects in sinus rhythm at 24 hours after start of study drug
(c) time to restoration of sinus rhythm within 24 hours

Statistical Considerations:
(a) Placebo subjects in all dose cohorts pooled to create one placebo dose group for comparison to the active dose groups
(b) each dose group compared separately with placebo
(c) Fisher's exact tests for difference in proportions between each dose level and placebo
(d) Time to restoration tested using the Kaplan-Meier method with difference in survivor functions; Log-Rank test used to compare each dose level with placebo
(e) No correction for multiple comparisons among dose groups.

TABLE 1
Atrial Fibrillation/Flutter history:

| | Placebo (32) | 200 mg (22) | 300 mg (25) | 400 mg (25) |
|---|---|---|---|---|
| A Flutter at | 4 (12.5) | 4 (18.2) | 4 (16) | 4 (16 |
| Entry N (%) | | | | |

Duration of Concurrent AF/AFL Episode

| | | | | |
|---|---|---|---|---|
| Mean, days | 1.84 | 2.33 | 2.43 | 1.97 |
| range, days | 0-6 | 0-6 | 0-6 | 0-7 |
| Rx same day as | 41 | 23 | 32 | 32 |
| onset, % | | | | |

Time since AF/AFL Dx

| | | | | |
|---|---|---|---|---|
| Mean, yrs | 3.9 | 4.8 | 4.5 | 5.1 |
| range, yrs | 0-21 | 0-13 | 0-13 | 1-13 |
| Rx prior DC | 44 | 45 | 52 | 32 |
| cadioversion % | | | | |

Time since last DC Cardioversion

| | | | | |
|---|---|---|---|---|
| Mean, mo | 13.6 | 15.2 | 18.2 | 21 |
| range, mo | 0-77 | 0-5 | 0-90 | 0-103 |

TABLE 2
Efficacy: Percent conversion through 4, 8, and 24 hours

| | Placebo (32) | 200 mg (22) | 300 mg (25) | 400 mg (25) |
|---|---|---|---|---|
| 0-4 hr | 13% | 18% | 40% | 52% |
| 0-8 hr | 23% | 45% | 52% | 76% |
| 0-24 hr | 38% | 59% | 64% | 84% |

Indeed, there is a significant improvement in conversion as compared to placebo at all time-points, wherein the rate of conversion or percent conversion at 0-4 hours, 0-8 hours and 0-24 hours was improved with any dose of vanoxerine. Accordingly, a measurement of the improvement comprises a comparison to the rate of conversion of placebo, wherein the improvement is based on the percent increase in conversion over placebo. The 200 mg, having an improvement of conversion of 38%, 96%, and 55% at the above time points, 300 mg: 207%, 126%, and 68%, and the 400 mg: 300%, 230%, and 121%.

TABLE 3
Time to conversion

| Log-rank test results for time conversion | P-value |
|---|---|
| Overall | 0.0005 |
| Pairwise: 200 mg versus control | 0.0838 |
| pairwise: 300 mg versus control | 0.0180 |
| pairwise: 400 mg versus control | <0.0001 |

Indeed, the time to conversion based on the P-value and the above chart provides that placebo does not have greater than a 40% conversion at any time point below 24 hours, whereas all doses of vanoxerine are greater than 40% conversion at about 7 hours, and conversion greater than 50% for all dose at 12 hours, and nearing 60% at about 16 hours.

TABLE 4
Conversion of Atrial Flutter

| | Placebo (32) | 200 mg (22) | 300 mg (25) | 400 mg (25) |
|---|---|---|---|---|
| A flutter, N | 4 | 4 | 4 | 4 |
| Conversion, % | 25% | 50% | 75% | 75% |

Definition of “pure” atrial flutter: only Atrial Flutter (no AF) seen at −30, −15, and 0 time points. Conversion at any time within 24 hours. No 1:1 AFL seen post dose in any subject.

TABLE 5
Adverse events:

| Placebo (32) | 200 mg (22) | 300 mg (25) | 400 mg (25) |
|---|---|---|---|
| 7 (22%) | 4 (18%) | 7 (28%) | 10 (40%) |
| subjects | subjects | subjects | subjects |
| reporting | reporting | reporting | reporting |
| 10 AE's | 8 AEs (1 SAE) | 12 AEs | 23 AEs (1 SAE) |

In view of doses of 200, 300 and 400 mg, there was a highly statistically significant dose dependent increase in the conversion to sinus rhythm of recent onset symptomatic AF/AFL. The highest oral dose of 400 mg achieved a conversion rate of 76% at 8 hours and 84% within 24 hours. Time to conversion curves also demonstrate increasing slope of conversion with successively higher doses, suggesting a Cₘₐₓ dependent effect.

Vanoxerine was well tolerated at all doses with only two serious adverse events, one at the 200 mg dose and one at the 400 mg dose (the 200 mg dose being an upper respiratory infection, the 400 mg dose being lower extremity edema secondary to amlodipine), neither related to the study drug. Similar to efficacy, there was a dose dependent increase in adverse events, but only the high dose event rate was notably higher than that of the placebo group. Accordingly, vanoxerine has a high degree of efficacy for the conversion of recent onset symptomatic atrial fibrillation and atrial flutter in the absence of proarrhythmia, wherein the conversion rate approaches that of DC cardioversion.

Accordingly, vanoxerine has a high degree of efficacy for the conversion of recent onset symptomatic atrial fibrillation and atrial flutter in the absence of proarrhythmia, wherein the conversion rate approaches that of DC cardioversion.

### Example 2

12 subjects received daily doses of vanoxerine for 11 consecutive days, at doses of 25, 50, 75, and 100 mg, with a 14 day washout period between dose levels.

At 25 mg, plasma levels were not detectable after 8 hours. At 50, 75, and 100 mg doses, plasma levels were detectable at 24 hours and steady state was reached by day 8. PK was linear and dose proportional across 50, 75 and 100 mg doses. The 100 mg QD Cₘₐₓₛₛ and AUC₀₋₂₄ₛₛ suggests a trend toward non-linear PK that may become apparent at doses >100 mg QD. PK was highly variable at steady state; Cₘₐₓ, ss, and AUC₀₋₂₄ₛₛ inter-subject variability ranged from 55-85%. The results are listed below in Table 6.

TABLE 6

| | PK Data | PK Data |
|---|---|---|
| Dose | (Mean +/− SD) C_{Max} | (Mean +/− SD) T_{1/2} |
| 50 mg | 27.5 +/ 21.3 ng/ml | 49.39 +/− 26.18 hr |
| T_{Max} 1.27 +− 0.5 hr | | (4.71-110.57) |
| (0.5-2.0) | | |
| 75 mg | 27.4 +/− 15.5 ng/ml | 52.53 +/− 37.46 |
| | | (10.26-116.67) |
| 100 mg | 40.2 +/− 26.6 ng/ml | 15.38 +/− 43.55 |
| | | (5.56-125.00) |

Data from these studies demonstrates an increased half-life of the drug when daily doses are given. Furthermore, it was noted that heart rate and systolic blood pressure increased slightly in most subjects at 75 and 100 mg doses and did not completely return to baseline during washout between dose levels.

### Example 3

Fourteen healthy patients were given vanoxerine of 25, 75, and 125 mg, daily, for 14 days with a washout of 14 days between dose levels. A standardized meal was served 15 minutes prior to each dosing.

No significant adverse events were seen in any of the studies. Steady state serum levels were reported within 9-11 days with disproportionately and statistically greater levels at higher doses as compared with the lower doses. The non-linear kinetics may be due to increasing bioavailability at higher doses based on a saturation of first pass metabolism.

### Example 4

Four patients were given 50, 100, and 150 mg vanoxerine, daily, for 7 days.

Upon administration of 100 mg for 7 days, increases in systolic blood pressure and heart rate were seen. Similarly, during the 150 mg test, the patients also saw increases in systolic blood pressure and in heart rate. Steady state levels were achieved within one week for all patients

Accordingly, hemodynamic effects on heart rate and systolic blood pressure have been seen with multiple dosing of vanoxerine. Several subjects exhibited dose-related increases in heart rate and systolic blood pressure. These effects, however, do not correlate with vanoxerine concentration AUC and interpretation is further confounded by the lack of placebo-control. These effects do not immediately dissipate upon discontinuation of study drug. It is suggested that vanoxerine exerts an effect on the autonomic nervous system over the course of the study. The lack of correlation with plasma vanoxerine AUC, may be interpreted as either evidence of a significant pharmacodynamic lag in the hemodynamic effects of vanoxerine or evidence that a metabolite is responsible for the hemodynamic effects.

Accordingly, because of the long half-life, a method of administration of dosing vanoxerine comprises an initial dose of 200-400 mg of vanoxerine sufficient to restore normal sinus rhythm, followed by a loading dose of the drug until steady-state concentration is met followed by subsequent administration about every 24, 48, or 72 hours to maintain therapeutic blood levels without the adverse effects of increased systolic blood pressure or heart rate.

In particular, it may be advantageous to determine the profile of the patient because of the known variability with vanoxerine such that the schedule for subsequent administration of vanoxerine post the loading phase is determined by the pharmacokinetic profile of the individual patient. In view of the studies, repeated dosing at 200 mg and above suggests that side effects may be prohibitive. However, an initial single dose greater than 200 mg provides a significant and tangible benefit of immediate reduction of symptoms and return to normal sinus rhythm as compared to placebo, with regard to treatment of recent onset AF and AFL. Accordingly, wherein repeated higher doses may not be practical, single doses may be particularly effective for symptomatic treatment in patients.

Accordingly, a method comprises administration of a single dose of vanoxerine of between 200 to 400 mg, to a patient to restore normal sinus rhythm. Upon reaching normal sinus rhythm, a medical professional can determine whether further administration is necessary, and may accordingly induce steady state status, through subsequent daily administration for 3-14 total days. Upon reaching steady state status, a maintenance regimen comprising administration of vanoxerine as necessary to maintain therapeutic steady-state levels is maintained so as to help prevent re-occurrence of the arrhythmia.

In some embodiments, the steady state administration and subsequent maintenance regimen may be instituted through the use of an infusion device that provides the appropriate dose to the patient on a regular basis. However, other suitable mechanisms, dosing schedules, and administration strategies are suitable for the initial dose, the dose or doses to induce steady state status, and for the maintenance doses so as to maintain the steady-state status in the patient. For example, blister packs may assist in appropriate doses during the loading phase, so as to achieve steady state, and during the maintenance phase. Blister packs organize pills, and may advantageously include placebo pills to appropriately spread out doses of vanoxerine, or include other medications that may advantageously be administered to the patient.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A method for restoring normal sinus rhythm to a patient suffering from recent onset symptomatic atrial fibrillation or atrial flutter in less than about 24 hours by administering to said patient at least 200 mg of vanoxerine.

2. The method of claim 1 comprising at least 300 mg of vanoxerine.

3. The method of claim 1 comprising at least 400 mg of vanoxerine.

4. The method of claim 1 wherein the patient is returned to normal sinus rhythm in less than about 12 hours.

5. The method of claim 1 wherein the patient is returned to normal sinus rhythm in less than about 8 hours.

6. The method of claim 1 wherein the patient is returned to normal sinus rhythm in less than about 4 hours.

7. A method of treating a patient suffering from symptoms of atrial fibrillation or atrial flutter for less than about 72 hours comprising administration of about 200 to about 400 mg of vanoxerine.

8. The method of claim 7 wherein said patient is suffering from atrial fibrillation or atrial flutter symptoms for less than about 48 hours.

9. The method of claim 7 wherein said patient is suffering from atrial fibrillation or atrial flutter symptoms for less than about 24 hours.

10. The method of claim 7 wherein said patient is returned to normal sinus rhythm in less than about 24 hours.

11. The method of claim 7 wherein said patient is returned to normal sinus rhythm in less than about 8 hours.

12. A method of treating a patient having recent onset of AF or AFL comprising administration of a single dose of a pharmaceutical composition comprising about 200 to about 400 mg of vanoxerine, and wherein said patient is converted to normal sinus rhythm at a rate of at least 33% better than conversion as compared to placebo at a time period of 0-4 hours.

13. The method of claim 12 wherein the time period is from 0-8 hours.

14. The method of claim 12 wherein the time period is from about 0-24 hours.

15. The method of claim 12 wherein the conversion is at least 50% better than conversion as compared to placebo at a time period of 0-4 hours.

16. The method of claim 12 wherein the conversion is at least 50% better than conversion as compared to placebo at a time period of 0-8 hours.

17. The method of claim 12 wherein the conversion is at least 50% better than conversion as compared to placebo at a time period of 0-24 hours.

18. The method of claim 12 wherein the conversion is at least 100% better than conversion as compared to placebo at a time period of 0-4 hours.

19. The method of claim 12 wherein the conversion is at least 100% better than conversion as compared to placebo at a time period of 0-8 hours.

20. The method of claim 12 wherein the conversion is at least 100% better than conversion as compared to placebo at a time period of 0-24 hours.

21. A method for terminating atrial flutter or atrial fibrillation comprising:
a. administering a first dose of at least 200 mg of vanoxerine to a patient to terminate said atrial flutter or atrial fibrillation in less than about 24 hours;
b. administering a subsequent dose of an effective amount of vanoxerine to achieve steady state in the patient; and
c. administering an effective amount of vanoxerine to maintain steady state in the patient.
